(19) United States
(12) Patent Application Publication
(10) Pub. No.: US 2019/0270936 A1
(43) Pub. Date: Sep. 5, 2019

(54) CATALYTIC LIQUEFACTION (CTL) METHOD FOR PRODUCTION OF BIO-CRUDE OIL USING IONIC LIQUID CATALYST AND PREPARATION THEREOF

(71) Applicant: Arvind Mallinath Lali, Mumbai (IN)

(72) Inventors: Arvind Mallinath LALI, Mumbai (IN); Hitesh PAWAR, Mumbai (IN); Sreenivasan SHRAVAN, Mumbai (IN)

(21) Appl. No.: 16/320,569

(22) PCT Filed: Jul. 25, 2017
(86) PCT No.: PCT/IN2017/050303

§ 371 Date: Jan. 25, 2019

(30) Foreign Application Priority Data
Jul. 25, 2016 (IN) 201621025317

(51) Int. Cl. C10G 1/04; C10G 1/08

(52) U.S. Cl. CPC C10G 1/042; C07C 211/14; C10G 1/086

(57) ABSTRACT

The present disclosure relates to a sulfonate-based ionic liquid. A simple process for obtaining the ionic liquid is provided. The conversion of waste into a usable bio-crude oil via a liquefaction process is further described, where the ionic liquid is employed as a catalyst.

### TECHNICAL FIELD

The present invention relates to a method for synthesis of ionic liquid catalyst. Furthermore, it relates to catalytic liquefaction (CTL) method for production of bio-crude oil from waste using said ionic liquid catalyst.

### BACKGROUND

The world energy consumption has been steadily increasing from last few decades due to a variety of reasons like enhancements in quality of life, population increase, industrialization, rapid economic growth of developing countries, increased transportation of people and goods, etc. Thus, as a global community, we are facing the challenge of increasing energy consumption, dwindling fossil fuel reserves, and the growing consequences of climate change. It is anticipated that by 2035 world's energy consumption is expected to increase over 50%. In order to meet the rising energy demand, researchers have focused their attention towards bio based materials such as biomass, municipal solid waste (MSW) and algae, as a potential substitute for producing renewable fuels and chemicals. Not only their wide spread and easily availability but also utilization of these bio-based materials help in maintaining the carbon balance on the surface of earth.

With the growing concerns over the environmental and health concerns associated with solid waste accumulation, the safe disposal of waste has been a major issue over the last few years. For e.g., the municipal solid waste management (MSWM) has been a major environmental issue in developing countries like India. The key issues relating to the management of MSW is related to the reasons such as: (1) absence of comprehensive short and long-term plan for disposal of MSW; (2) awareness to set up waste processing and disposal facilities. Hence, recycling of solid waste into useful products helps solve the additional problem of waste accumulation. The liquefaction of solid waste into bio-crude oil is a dynamic approach, which could potentially solve the growing energy crisis and waste accumulation problems in one stroke. As an example, the MSW majorly constitutes of organic, inorganic and ceramic materials waste. The recoverable carbon rich fraction amongst this is the biodegradable waste matter (organic matter) which amounts to around 75% in MSW.

Liquefaction of MSW is a promising scientific solution to treat the MSW into energy dense “bio-crude” fuel or oil that can be used for direct combustion or refined for transportation grade fuels. Several methods, for example, hydrothermal liquefaction (HTL) and hydrothermal upgrading processes (HTU) have been reported for conversion of MSW into liquid bio-crude oil. In the hydrothermal upgrading process (HTU), developed by Shell, biomass is treated with water at high temperature and pressure (300-350° C. and 120-180 bar) to produce bio-crude. A number of such reports have been published (Biomass and Bioenergy, 1998, 14, 5-6; PNNL-2014; US 20130331623; US 20120005949; U.S. Pat. No. 9,006,502), however, much like the report by Shell, these suffer from the drawback of requirement of high temperature and high pressure. The catalytic liquefaction of biomass using precious metals has provided greater yields (Algal Research, 2013, 2, 455-464; Industrial and Engineering Chemistry Research, 2010, 50, 1, 52-61; Appl. Energy 2012, 98, 368-375), but the cost effectiveness of such formulations, especially considering the additional requirement of high temperature, is questionable. Hence, there is a need to identify a low temperature, ambient pressure method for liquefaction of waste resulting in production of high yield bio-crude oil.

### Objects of Present Invention

The principle object of the present invention is to provide a sulfonic acid based ionic liquid which are easier to separate and reuse.

In another object of the present invention is a method for synthesis of sulfonic acid counter ion Bronsted acid ionic liquid (IL) catalyst for production of bio-crude oil with complete conversion and higher yield.

Another object of present invention is to provide a catalytic liquefaction (CTL) method for conversion of organic biodegradable waste into energy dense bio-crude oil using said ionic liquid catalyst at non-stringent reaction condition and also provides recovery and recycling of ionic liquid catalyst with complete conversion of waste into bio-crude oil which can be used as energy source or converted into value added product.

### SUMMARY

One of the aspects of the present invention is to provide a sulfonic acid-based ionic liquid.

In another aspects of present invention is to provide a method for synthesis of sulfonic acid counter ion Bronsted acid ionic liquid (IL) catalyst, wherein said method comprises of: (a) contacting aromatic or aliphatic nitrogen substrate with sulfonating agent in presence of solvent to form a reaction mixture; (b) stirring the said reaction mixture to form suspension of quaternary nitrogen counter ion Bronsted acid ionic liquid catalyst; (c) isolating quaternary nitrogen sulfonic acid counter ion Bronsted acid ionic liquid catalyst.

Another aspect of present invention is to provide a catalytic liquefaction (CTL) method for production of bio-crude oil from waste using ionic liquid (IL) catalyst. In the CTL method organic-biodegradable waste is sorted from non-biodegradable waste. The size of organic biodegradable waste is reduced to obtain uniform particle size for waste slurry preparation. The said slurry is made in water or water miscible organic solvent and then contacted with indigenously prepared ionic liquid catalyst. The composite slurry is heated at defined temperature for sufficient time to obtain bio-crude oil. During the formation of bio-crude oil, complex biopolymeric components in the waste are broken down into non-complex/monomeric organic compounds. The unreacted waste and ionic acid catalyst are separated from bio-crude oil and recycled for next CTL for complete conversion into bio-crude oil. Bio-crude oil is extracted from aqueous phase by using water immiscible organic solvent and isolated by distillation organic solvent.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively, and any and all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. Throughout this specification, unless the context requires otherwise the word “comprise”, and variations, such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “at least one” is used to mean one or more and thus includes individual components as well as mixtures/combinations.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a temperature ranges of about 25-40° C. should be interpreted to include not only the explicitly recited limits of about 25° C. to about 40° C., but also to include sub-ranges, such as 25-30° C., 28-38° C., and so forth, as well as individual amounts, including fractional amounts, within the specified ranges, such as 25.2° C., and 38.5° C., for example.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific implementations described herein, which are intended for the purposes, of exemplification only. Functionally-equivalent products, compositions, and methods are clearly within the scope of the disclosure, as described herein.

The term “Waste/s” used herein refers to unwanted or unusable materials. Waste is any substance which is discarded after primary use, or it is worthless, defective and of no use. Wastes may be generated during the extraction of raw materials, the processing of raw materials into intermediate and final products, the consumption of final products, and other human activities. The waste material includes but is not limited to municipal solid waste, food waste, kitchen waste, vegetable waste, paper waste, poultry waste, fruit waste, house hold waste, agriculture and forestry waste. The term “waste/s” may be used singularly or plurally in the specification.

The term ‘nitrogen pre-substrate’ refers to a compound selected from the group consisting of five or six membered nitrogen containing aromatic compounds or their derivatives in which nitrogen is present in the form of free or ring primary (1°) and/or secondary (2°) and/or tertiary (3°) amine group. For e.g. imidazole.

The term “catalytic liquefaction (CTL)” used herein refers to process for liquefaction of waste/s into energy dense bio-crude oil in presence of ionic liquid catalyst at non-stringent reaction conditions.

The term “ionic liquid (IL)” used herein as a catalyst refers to sulfonic acid counter ion Bronsted acid ionic liquid having aliphatic or aromatic nitrogen containing backbone molecule. The terms “ionic liquid (IL)” and “ionic liquid (IL) catalyst” are used interchangeably in the specification.

For example

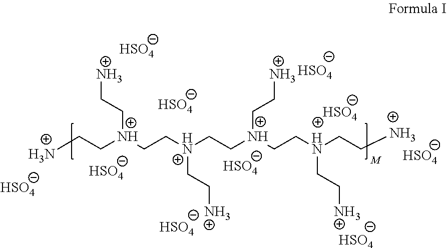

wherein M is number average molecular weight in the range of 0.2-150 KD, and

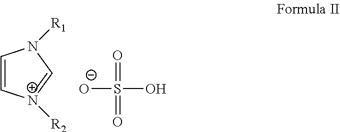

wherein R1 is selected from the group consisting of C₁ to C₁₀ alkyl and C₁ to C₂₀ aryl, wherein the alkyl and aryl are optionally substituted with halo, —OH, aryl and alkyl;
R2 is selected from the group consisting of hydrogen, HSO₃⁻, C₁ to C₁₀ alkyl and C₁ to C₂₀ aryl.

The term “waste slurry” OR “slurry” used herein refers to mixture of size reduced waste material and water or water miscible organic solvent. The terms “waste slurry” OR “slurry” can be used interchangeably in the specification.

The term “bio-crude fuel” used herein refers to the liquid or oil obtained by liquefaction of waste selected from the group but not limited to municipal solid waste garbage, lignocellulosic biomass, algae, road swipe, kitchen waste, vegetable waste, cook food waste, paper waste, garden waste etc. It is used interchangeably with “bio-crude oil”.

The term “reaction mixture” used herein refers to a mixture in which one or more substance of waste slurry reacts with catalyst to form reaction product.

The term “[BenzIMSO₃H⁺][HSO₄⁻]” used herein refers to Bronsted acid ionic liquid having imidazole backbone having the structure represented by Formula II, wherein R1 is —CH₂C₆H₅ (benzyl or Bz); and R2 is HSO₃⁻. The term “[PEI⁺][HSO₄⁻]” used herein refers to Bronsted acid ionic liquid having polyethylene imine backbone and sulfonic acid counter ion having the structure represented by Formula I.

Following paragraphs describe the invention in detail, elaborating upon various aspects of the invention. The existing HTL processes reported by several academia and industry researchers suffer from several economical hurdles as, (1) use of high temperature and pressure, (2) use of precious metal catalyst, (3) low bio crude yield with maximum char formation, (4) stringent reaction and workup process, (5) engineering challenges for designing reactor systems etc. Hence the development of a catalytic process that allows the convenient conversion of solid waste into useful bio-fuel is of utmost importance. In the present invention, there is disclosed a new strategy to convert the organic biodegradable components of waste into energy dense bio-crude oil via catalytic liquefaction (CTL) method using novel sulfonic acid counter ion Bronsted acid ionic liquid as catalyst. The process was found to have high yield of bio-crude oil (greater than 80%) and conversion (greater than 85%) at non stringent reaction conditions.

In an embodiment of the present invention there is provided an ionic liquid selected from the group consisting of a compound of Formula I having viscosity in the range of 100-2000 cpi (centipoise) at 30° C.

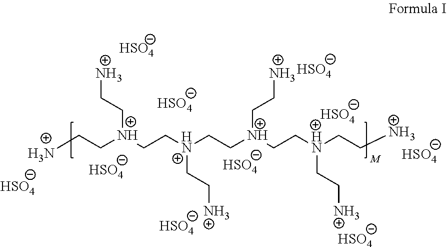

wherein M is number average molecular weight in the range of 0.2-150 KD; and
a compound of Formula II having viscosity in the range of 100-2000 cpi at 30° C.

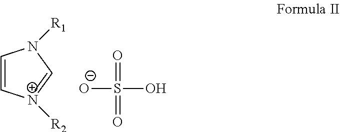

wherein R1 is selected from the group consisting of C₁ to C₁₀ alkyl and C₁ to C₂₀ aryl, wherein the alkyl and aryl are optionally substituted with halo, —OH, aryl and alkyl;
R2 is selected from the group consisting of hydrogen, HSO₃⁻, C₁ to C₁₀ alkyl and C₁ to C₂₀ aryl, wherein the alkyl and aryl are optionally substituted with halo, —OH, aryl and alkyl.

In an embodiment of the present there is provided an ionic liquid herein, wherein the ionic liquid is represented by Formula I having viscosity in the range of 100-2000 cpi (centipoise) at 30° C.

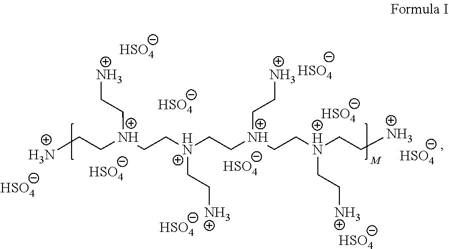

wherein M is number average molecular weight in the range of 0.2-150 KD. The said ionic liquid is represented with the term [PEI⁺] [HSO₄⁻].

In an embodiment of the present there is provided an ionic liquid herein, wherein the ionic liquid is represented by formula II having viscosity in the range of 100-2000 cpi at 30° C.

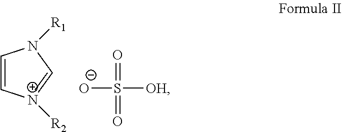

wherein R1 is selected from the group consisting of C₁ to C₁₀ alkyl and C₁ to C₂₀ aryl, wherein the alkyl and aryl are optionally substituted with halo, —OH, aryl and alkyl;
R2 is selected from the group consisting of hydrogen, HSO₃⁻, C₁ to C₁₀ alkyl and C₁ to C₂₀ aryl, wherein the alkyl and aryl are optionally substituted with halo, —OH, aryl and alkyl.

In an embodiment of the present there is provided an ionic liquid herein, wherein the ionic liquid is represented by formula II having viscosity in the range of 100-2000 cpi at 30° C.

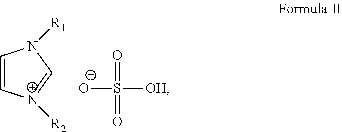

wherein R1 is selected from the group consisting of C₁ to C₃ alkyl, wherein the alkyl is optionally substituted with C₅ to C₁₀ aryl; R2 is HSO₃⁻.

In an embodiment of the present there is provided an ionic liquid herein, wherein the ionic liquid is represented by formula V having viscosity in the range of 100-2000 cpi at 30° C.

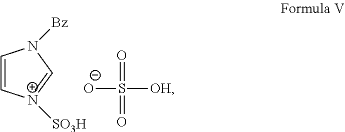

wherein R1 is —CH₂C₆H₅ (benzyl or Bz); and R2 is HSO₃⁻. The said ionic liquid is represented with the term [BenzIMSO₃H⁺] [HSO₄⁻].

In an embodiment of the present invention there is provided a process for obtaining an ionic liquid as described herein, the process comprising the steps of: a) contacting at least one nitrogen substrate with at least one sulfonating agent and optionally at least one solvent to obtain a first mixture; b) stirring the first mixture to obtain ionic liquid; and c) isolating the ionic liquid.

In an embodiment of the present invention there is provided a process for obtaining ionic liquid as described herein, wherein the at least one nitrogen substrate is a compound of Formula III

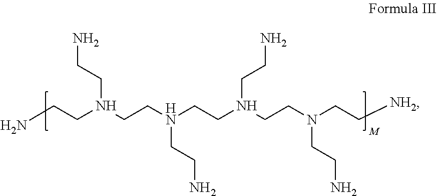

wherein M is number average molecular weight in the range of 0.2-150 KD. In another embodiment of the present invention the process comprising the steps of: a) contacting the compound of Formula III with at least one sulfonating agent and at least one solvent to obtain a first mixture; b) stirring the first mixture to obtain the ionic liquid; and c) isolating the ionic liquid.

In an embodiment of the present invention there is provided a process for obtaining ionic liquid as described herein, wherein the at least one nitrogen substrate is a compound of Formula IV

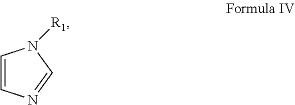

wherein R1 is selected from the group consisting of C₁ to C₁₀ alkyl and C₁ to C₂₀ aryl, wherein the alkyl and aryl are optionally substituted with halo, —OH, aryl and alkyl. In another embodiment of the present invention the process comprising the steps of: a) contacting the compound of Formula IV with at least one sulfonating agent and optionally at least one solvent to obtain a first mixture; b) stirring the first mixture to obtain the ionic liquid; and c) isolating the ionic liquid.

In an embodiment of the present invention there is provided a process for obtaining ionic liquid as described herein, wherein the at least one nitrogen substrate is a compound of Formula IV

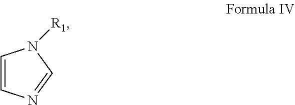

wherein R1 is selected from the group consisting of C₁ to C₃ alkyl, wherein the alkyl is optionally substituted with C₅ to C₁₀ aryl. In another embodiment of the present invention the nitrogen substrate is a compound of Formula VI

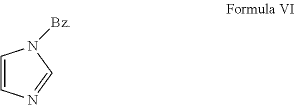

In another embodiment of the present invention the process comprising the steps of: a) contacting the compound of Formula VI with at least one sulfonating agent and optionally at least one solvent to obtain a first mixture; b) stirring the first mixture to obtain the ionic liquid; and c) isolating the ionic liquid.

In an embodiment of the present invention there is provided a process for obtaining ionic liquid as described herein, wherein at least one solvent is selected from the group consisting of dichloromethane, ethylene dichloride, chloroform, carbon tetrachloride and combinations thereof.

In an embodiment of the present invention there is provided a process for obtaining ionic liquid as described herein, wherein isolating the ionic liquid is carried out by a process selected from the group consisting of filtration, distillation, organic extraction, evaporation, chromatographic separation, and combinations thereof.

In an embodiment of the present invention there is provided a process for obtaining ionic liquid as described herein, wherein stirring the first mixture is carried out a temperature in the range of 30-150° C. at a stirring speed in the range of 400 rpm for a period in the range of 15-120 min. In another embodiment of the present invention the stirring the first mixture is carried out at a temperature in the range of 30-80° C. at a stirring speed in the range of 400 rpm for a period in the range of 15-120 min. In yet another embodiment of the present invention the stirring the first mixture is carried out at a temperature in the range of 30-150° C. at a stirring speed in the range of 400 rpm for a period in the range of 15-120 min.

In an embodiment of the present invention there is provided a process for obtaining ionic liquid as described herein, wherein the process comprising the steps of: a) contacting at least one nitrogen substrate with at least one sulfonating agent and optionally at least one solvent to obtain a first mixture; b) stirring the first mixture is carried out a temperature in the range of 30-150° C. at a stirring speed in the range of 400 rpm for a period in the range of 15-120 min to obtain the ionic liquid; and c) isolating the ionic liquid.

In an embodiment of the present invention there is provided a process for obtaining ionic liquid as described herein, wherein the process comprising the steps of: a) contacting the compound of Formula III with sulfuric acid to obtain a first mixture; b) stirring the first mixture is carried out at a temperature in the range of 30-80° C. at a stirring speed in the range of 400 rpm for a period in the range of 15-120 min to obtain the ionic liquid; and c) isolating the ionic liquid.

In an embodiment of the present invention there is provided a process for obtaining ionic liquid as described herein, wherein the process comprising the steps of: a) contacting the compound of Formula IV with sulfuric acid and dichloromethane to obtain a first mixture; b) stirring the first mixture is carried out at a temperature in the range of 30-150° C. at a stirring speed in the range of 400 rpm for a period in the range of 15-120 min to obtain the ionic liquid; and c) isolating the ionic liquid.

In an embodiment of the present invention there is provided a process for obtaining ionic liquid as described herein, wherein the process comprising the steps of: a) contacting the compound of Formula VI with sulfuric acid and dichloromethane to obtain a first mixture; b) stirring the first mixture is carried out at a temperature in the range of 30-150° C. at a stirring speed in the range of 400 rpm for a period in the range of 15-120 min to obtain the ionic liquid; and c) isolating the ionic liquid.

In an embodiment of the present invention there is provided a process for obtaining ionic liquid as described herein, wherein the process comprising the steps of: a) optionally contacting imidazole with benzylchloride and toluene is carried out a temperature in the range of 100-120° C. at a stirring speed in the range of 400 rpm for a period in the range of 180 min to obtain the compound of Formula VI b) contacting the compound of Formula VI with sulfuric acid and dichloromethane to obtain a first mixture; b) stirring the first mixture is carried out at a temperature in the range of 30-150° C. at a stirring speed in the range of 400 rpm for a period in the range of 15-120 min to obtain the ionic liquid; and c) isolating the ionic liquid.

In an embodiment of the present invention there is provided a process for obtaining ionic liquid as described herein, wherein the process comprising the steps of: (1) optionally synthesizing nitrogen substrate (2) stoichiometric mixing of nitrogen substrate with mineral or organic acid (3) reacting nitrogen substrate with mineral or organic acid to form corresponding quaternary nitrogen counter ion Bronsted acid ionic liquid; (4) isolating said quaternary nitrogen sulfonic acid counter ion Bronsted acid ionic liquid.

In an embodiment of the present invention there is provided a process for obtaining ionic liquid as described herein, wherein optionally synthesizing nitrogen substrate is carried out by treating a nitrogen pre-substrate with alkylating agent. In another embodiment of the present invention, the alkylating agent is selected from the group consisting of C₁ to C₁₀ alkyl halides and C₁ to C₂₀ aryl halides, wherein the alkyl halide and aryl halide are optionally substituted with halo, —OH, aryl and alkyl. In yet another embodiment of the present invention the nitrogen pre-substrate is selected from the group consisting of five or six membered nitrogen containing aromatic compounds or their derivatives in which nitrogen is present in the form of free or ring primary (1°) and/or secondary (2°) and/or tertiary (3°) amine group. In another embodiment of the present invention the alkylating agent is benzyl chloride and the nitrogen pre-substrate is imidazole.

In an embodiment of the present invention there is provided a process for obtaining ionic liquid as described herein, wherein the nitrogen substrate used is a five or six membered nitrogen containing aromatic compounds or their derivatives in which nitrogen is present in the form of free or ring primary (1°) and/or secondary (2°) and/or tertiary (3°) amine group.

In an embodiment of the present invention there is provided a process for obtaining ionic liquid as described herein, wherein the nitrogen substrate used is a five or six membered nitrogen containing aliphatic compounds or their derivatives or aliphatic nitrogen containing polymers in which nitrogen is present in the form of free or ring 1° or 2° or 3° amine group such as but not limited to e.g. morpholine, ethylenediamine, polyethyleneimine, and combinations thereof.

In an embodiment of the present invention there is provided a process for obtaining ionic liquid as described herein, wherein the aliphatic nitrogen substrate used may be in the form of linear or branched or cross linked amino nitrogen containing polymers or their derivatives.

In an embodiment of the present invention there is provided a process for obtaining ionic liquid as described herein, wherein the nitrogen substrate used is linear or branched amino nitrogen containing polymers selected from but not limited to the molecular weight in the range of 0.2-150 KD.

In an embodiment of the present invention there is provided a process for obtaining ionic liquid as described herein, wherein the nitrogen substrate is treated with an acid. The mineral or organic acid used for forming corresponding quaternary nitrogen counter ion Bronsted acid ionic liquid is selected from the group H₂SO₄, H₃PO₄, CH₃COOH, PTSA, and combinations thereof.

In an embodiment of the present invention there is provided a process for obtaining ionic liquid as described herein, wherein the sulfonating agent is selected from the group consisting of chlorosulfonic acid, sulphuric acid, sulfur trioxide, and combinations thereof. In another embodiment of the present invention, the sulfonating agent is sulfuric acid. In yet another embodiment of the present invention, the sulfonating agent is chlorosulfonic acid.

In an embodiment of the present invention there is provided a process for obtaining a bio-crude fuel comprising the steps of: a) contacting a waste with water to form a first mixture; b) contacting the first mixture with at least one ionic liquid as described herein; c) heating the first mixture to obtain a second mixture; d) processing the second mixture to obtain the bio-crude fuel; and e) recovering the at least one ionic liquid from the second mixture to obtain recovered ionic liquid.

In an embodiment of the present invention there is provided a process for obtaining a bio-crude fuel as described herein, wherein the said ionic liquid is a macromolecule having molecular weight in the range of 0.2-150 KD and the said process is effective in recovering the ionic liquid from second reaction mixture by a process selected from the group consisting of membrane separation, selective precipitation, distillation, adsorptive separation, and combinations thereof.

In an embodiment of the present invention there is provided a process for obtaining a bio-fuel as described herein, wherein heating the first mixture is carried out at a temperature in the range of 30-200° C. at a stirring speed in the range of 300-450 rpm for a period in the range of 5-200 min.

In an embodiment of the present invention there is provided a process for obtaining a bio-crude fuel comprising the steps of: a) contacting a waste with water to form a first mixture; b) contacting the first mixture with the compound of the Formula I; c) heating the first mixture is carried out at a temperature in the range of 30-200° C. at a stirring speed in the range of 300-450 rpm for a period in the range of 5-200 min to obtain a second mixture; d) organic extraction of second mixture to obtain the bio-crude fuel; and e) recovering the compound of Formula I from the second mixture by methods selected from membrane separation, adsorptive separation, distillation and selective precipitation to obtain recovered ionic liquid.

In an embodiment of the present invention there is provided a process for obtaining a bio-crude fuel comprising the steps of: a) contacting a waste with water to form a first mixture; b) contacting the first mixture with the compound of the Formula I; c) heating the first mixture is carried out at a temperature of 120° C. at a stirring speed of 400 rpm for a period of 120 min to obtain a second mixture; d) organic extraction of second mixture to obtain the bio-crude fuel; and e) recovering the compound of the Formula I from the second mixture by membrane separation to obtain recovered ionic liquid.

In an embodiment of the present invention there is provided a process for obtaining a bio-crude fuel comprising the steps of: a) contacting a waste with water to form a first mixture; b) contacting the first mixture with the compound of the Formula II; c) heating the first mixture to obtain a second mixture; d) processing the second mixture to obtain the bio-crude fuel; and e) recovering the compound of the Formula II from the second mixture to obtain recovered ionic liquid.

In an embodiment of the present invention there is provided a process for obtaining a bio-crude fuel comprising the steps of: a) contacting a waste with water to form a first mixture; b) contacting the first mixture with the compound of the Formula I or II or V; c) heating the first mixture to obtain a second mixture; d) processing the second mixture to obtain the bio-crude fuel; and e) recovering the compound of the Formula I or II or V from the second mixture to obtain recovered ionic liquid.

In an embodiment of the present invention there is provided a process for obtaining a bio-crude fuel comprising the steps of: a) contacting a waste with water to form a first mixture; b) contacting the first mixture with the compound of the Formula V; c) heating the first mixture to obtain a second mixture; d) processing the second mixture to obtain the bio-crude fuel; and e) recovering the compound of the Formula V from the second mixture to obtain recovered ionic liquid.

In an embodiment of the present invention there is provided a process for obtaining a bio-crude fuel comprising the steps of: a) contacting a waste with water to form a first mixture; b) contacting the first mixture with the compound of the Formula V; c) heating the first mixture is carried out at a temperature of 120° C. at a stirring speed of 400 rpm for a period of 90 min to obtain a second mixture; d) processing the second mixture to obtain the bio-crude fuel; and e) recovering the compound of the Formula V from the second mixture by membrane separation to obtain recovered ionic liquid.

In an embodiment of the present invention there is provided a process for obtaining a bio-crude fuel comprising the steps of: a) contacting a waste with water to form a first mixture; b) contacting the first mixture with the compound of the Formula I or II or V; c) heating the first mixture is carried out at a temperature of 120° C. at a stirring speed of 400 rpm for a period of 90 min to obtain a second mixture; d) processing the second mixture to obtain the bio-crude fuel; and e) recovering the compound of the Formula I or II or V from the second mixture by membrane separation or distillation to obtain recovered ionic liquid.

In an embodiment of the present invention there is provided a process for obtaining bio-crude fuel as described herein, wherein ionic liquid catalyst may be separated by any downstream process selected from the group consisting of filtration, distillation, extraction, membrane and adsorptive separation, chromatographic separation, selective precipitation and combinations thereof.

In an embodiment of the present invention there is provided a process for obtaining ionic liquid as described herein, wherein the ionic liquid can be used for acid catalyzed reaction such as but not limited to esterification condensation, dehydration, hydrolysis reaction, and combinations thereof.

In an embodiment of the present invention there is provided a process for obtaining bio-crude fuel as described herein, wherein the waste is optionally pre-processed by maceration, crushing, grinding, and combinations thereof.

In an embodiment of the present invention there is provided a process for obtaining bio-crude fuel as described herein, wherein said process comprising the steps of:
a) preparing waste slurry in water or water miscible solvent;
b) contacting waste slurry with indigenously prepared ionic liquid catalyst to obtain a first reaction mixture;
c) heating the first reaction mixture at a temperature in the range of 30° C. to 200° C. for time period in the range of 30 min to 150 min to obtain a reaction product comprising bio-crude oil, unconverted or unreacted waste and ionic liquid catalyst;
   wherein complex biopolymeric components of biodegradable waste get broken down into non-complex or monomeric organic compounds;
d) separating the unconverted waste from reaction product obtained from step (c) and recycling it in step (a);
e) extracting bio-crude oil from reaction product from step (c) by using water immiscible organic solvent to obtain organic solvent phase comprising bio-crude oil, and aqueous phase comprising ionic liquid catalyst, and nutrient element such as nitrogen (N), phosphorus (P), potassium (K);
f) separating organic solvent phase from aqueous phase;
g) isolating bio-crude oil from organic solvent phase by known conventional methods;
   wherein bio-crude oil is obtained greater than 50% yield;
h) recovering ionic liquid catalyst from aqueous phase and recycling in step (b); and
i) repeating step (a) to (h) for continuous production of bio-crude oil.

In an embodiment of the present invention there is provided a process for obtaining bio-crude fuel as described herein, wherein waste used is selected from the group but not limited to biodegradable waste algae biomass (micro algae, microalgae etc.), Kitchen waste (e.g. cook food waste, vegetable waste, green waste, paper), organic recyclable materials (e.g. paper, cardboard, glass, bottles, certain plastics, fabrics, clothes, tyres, etc.), composite wastes (e.g. waste clothing, tetra packs, waste plastics such as toys etc.), hazardous waste (e.g. paints, chemicals, tyres, fertilizers etc.), toxic waste (e.g. pesticides, herbicides, and fungicides), biomedical waste (e.g. expired pharmaceutical drugs, etc.), most preferably biodegradable waste.

In an embodiment of the present invention there is provided a process for obtaining bio-crude fuel as described herein, wherein waste slurry may be made in water or water miscible solvent or waste material has moisture content in the range of 10% to 90%. The water miscible solvent is selected from the group but not limited to alcohols, esters, ketones, acids, more preferably alcohols, ketones, most preferably alcohols.

In an embodiment of the present invention there is provided a process for obtaining bio-crude fuel as described herein, wherein said process is carried out by using ionic liquid catalyst. Said ionic liquid catalyst may be sulfonic acid counter ion Bronsted acid ionic liquid catalyst.

In an embodiment of the present invention there is provided a process for obtaining bio-crude fuel as described herein, wherein ratio of waste slurry to ionic liquid catalyst is in the range of 1:1 to 1:25 w/w on the basis of weight or dry weight.

In an embodiment of the present invention there is provided a process for obtaining bio-crude fuel as described herein, wherein the heating the first mixture is optionally carried out under pressure in the range of 0-50 bars. The said process may be carried out under inert gas pressure of 0 to 50 Kg/cm³ or at autogenous pressure or at reflux condition (boiling point temperature). The said inert gas used is selected from but not limited to nitrogen, argon, helium, air, and combinations thereof.

In an embodiment of the present invention there is provided a process for obtaining bio-crude fuel as described herein, wherein said method is carried out by heating the waste slurry with ionic liquid catalyst at a temperature in the range of 30° C. to 200° C., more preferably 50° C. to 150° C., most preferably 100° C. to 130° C. for the time period in the range of 5 min to 200 min, more preferably 50 min to 150 min, most preferably 60 min to 100 min under the stirring at agitation speed in the range of 300 rpm to 450 rpm.

In an embodiment of the present invention there is provided a process for obtaining ionic liquid as described herein, wherein the bio-crude fuel is obtained in a yield greater than 80%. In another embodiment of the present invention 80% to 100% conversion is obtained. In another embodiment of the present invention the conversion of at least 85% is achieved.

In an embodiment of the present invention there is provided a process for obtaining bio-crude fuel as described herein, wherein processing the second mixture is carried out by cooling the second mixture to room temperature. After cooling second mixture is diluted with sufficient amount of water and then filtered to remove unreacted waste. In yet another embodiment of present invention processing the second mixture may be carried out by any downstream process such as but not limited to filtration, extraction, distillation and chromatographic separation, membrane separation, selective precipitation, adsorptive separation and combinations thereof.

In an embodiment of the present invention there is provided a process for obtaining bio-crude fuel as described herein, wherein processing the second mixture to obtain bio-crude fuel is carried out by using methods such as but not limited to solvent extraction, distillation, membrane separation, or chromatographic separation etc. For the solvent extraction process the water immiscible solvent is selected from the group consisting of ethyl acetate, toluene, methylene dichloride, ethylene dichloride, acetonitrile or any non-polar solvent, and combination thereof. The water immiscible layer is separated from aqueous layer. The water immiscible solvent is distilled out to obtain bio-crude oil with yield greater than 80% and complete (100%) conversion. The aqueous layer contains nutrient elements such as nitrogen (N), phosphors (P), potassium (K), for use as a fertilizer or nutrient for cultivation of algae etc.

In an embodiment of the present invention there is provided a process for obtaining bio-crude fuel as described herein, wherein said process may be conducted in batch or continuous mode of operation for complete conversion of organic biodegradable wastes.

In an embodiment of the present invention there is provided a process for obtaining bio-crude fuel as described herein, wherein the bio-crude fuel obtained has calorific value in the range of 10 MJ/Kg to 35 MJ/Kg.

In an embodiment of the present invention there is provided a process for obtaining bio-crude fuel as described herein, wherein the bio-crude fuel is used directly as a fuel or as a fuel-additive in automobile, industrial and agricultural applications. In another embodiment of the present invention the bio-crude fuel can be used for value added products production, for heat generation, as transport fuel and/or fuel blend or for biogas generation.

Although the subject matter has been described in considerable details with reference to certain preferred embodiments thereof, other embodiments are possible.

The invention is further illustrated by working examples as detailed below. The examples are meant for illustrative purposes only and are not meant imply restriction to the scope of the disclosure in any manner.

### EXAMPLES

The following examples are given by way of illustration of the present invention and should not be construed to limit the scope of present disclosure. It is to be understood that both the foregoing general description and the following detailed description are exemplary and explanatory only and are intended to provide further explanation of the claimed subject matter.

Several conventional biochemical and thermochemical treatment methods have been studied in prior art to reclaim the energy from MSW. But available conventional methods of treating MSW are inefficient to reclaim the energy from MSW efficiently. In this regard, ionic liquids are dynamic molecules possessing several attractive qualities such as biodegradability, high polarity, ability to dissolve lipophilic molecules and negligible vapor pressure, which mark them out as potential catalysts. A convenient synthetic route to obtain these molecules is discussed below.

The following paragraphs discuss in detail the synthesis of ionic liquid and process for obtaining bio-crude fuel using the ionic liquid catalyst. The yields and conversion were calculated using the following formula—

$$\% Yield = \frac{Wt . of Biocrude Oil Isolated}{Therotical Wt of biocrude Oil} \times 100$$

$$\% Conversion = \frac{( Wt of initial solids in feed - Wt . of solid remains after CTL )}{( Wt . of initial solids in feed )} \times 100$$

### Example 1

### Synthesis of Ionic Liquid Catalyst

### (A) Imidazole Backbone Sulfonic Acid Counter Ion Bronsted Acid Ionic Liquid Catalyst

The imidazole based Bronsted acid ionic liquid synthesis was conducted in a four neck glass reactor equipped with reflux condenser, thermometer and overhead stirrer. Silicone oil bath, heated with electrical heating plate was used to heat the reaction reactor vessel. The reactor was fed with imidazole (1 Eq) and benzyl chloride (1 Eq) and heated to reflux in toluene for 3 hrs at 400 rpm. Then toluene was distilled completely and resulting viscous liquid (Yield>90%) was dissolved dichloromethane (DCM) (w/v) and again added in a four neck glass reactor equipped with reflux condenser, thermometer and overhead stirrer. The chlorosulfonic acid (1 Eq) was added slowly in 30 min and then reaction mixture was stirred for 1 hrs at 30° C. and 2 hrs at reflux temperature. Then DCM was removed by distillation to get viscous liquid (Yield. 90%) and then H₂SO₄ was added slowly for 30 min and resulting mixture was kept for 1 hrs under stirring to get imidazole based sulfonic acid counter ion Bronsted acid ionic liquid denoted as [BenzIMSO₃H⁺] [HSO₄⁻] (Yield>95%).

### (B) Polyethyleneimine (PEI) Back Bone Sulfonic Acid Counter Ion Bronsted Acid Ionic Liquid

The polyethyleneimine (PEI) based Bronsted acid ionic liquid synthesis was conducted in a four neck glass reactor equipped with reflux condenser, thermometer and overhead stirrer. Silicone oil bath, heated with electrical heating plate was used to heat the reaction reactor vessel. The reactor was fed with PEI (1 Eq) then sulfuric acid (1 Eq) was added slowly in 30 min and then reaction mixture was stirred for 1 hrs at 30° C. and 2 hrs at 50° C. temperature to get PEI based sulfonic acid counter ion Bronsted acid ionic liquid denoted as [PEI⁺] [HSO₄⁻] (Yield>95%).

### Example 2

### Catalytic Liquefaction of MSW for Production of Bio-Crude Oil

### (1) Production of Bio-Crude Oil Using Ionic Liquid Catalyst Synthesized in Example (1) (A) and Market Waste

The liquefaction reaction was conducted in a four neck glass reactor equipped with reflux condenser, thermometer and overhead stirrer. Silicone oil bath, heated with electrical heating plate was used to heat the reaction reactor vessel. The reactor was fed with 10 gm of crushed market MSW (H₂O content 70%) and [BenzIMSO₃H⁺] [HSO₄⁻] ionic liquid catalyst (1.13 times w/w of dry MSW) and was heated at 120° C. for 90 minutes at 400 rpm. After the completion of reaction, the vessel was allowed to cool to room temperature before opening reactor vessel. Sufficient amount of water was added to the reaction mixture followed by vacuum filtration to remove unreacted MSW. The resulting filtrate was then extracted two times with ethyl acetate and composite organic layer was collected and dried with NaCl and distilled to get bio-crude oil 2.57 gm (Yield>80%). The residue obtained after filtration was dried and the weight was noted to calculate percentage conversion (>85%). The calorific value of bio-crude oil from various MSW was 25 MJ/kg.

### (2) Production of Bio-Crude Oil Using Ionic Liquid Catalyst Synthesized in Example (1) (A) and Cook Food Waste

The liquefaction reaction was conducted in a four neck glass reactor equipped with reflux condenser, thermometer and overhead stirrer. Silicone oil bath, heated with electrical heating plate was used to heat the reaction reactor vessel. The reactor was fed with 10 gm of crushed cook food waste (H₂O content 70%) and [BenzIMSO₃H⁺] [HSO₄⁻] ionic liquid catalyst (1.13 times w/w of dry MSW) and was heated at 120° C. for 90 minutes at 400 rpm. After the completion of reaction, the vessel was allowed to cool to room temperature before opening reactor vessel. Sufficient amount of water was added to the reaction mixture followed by vacuum filtration to remove unreacted MSW. The resulting filtrate was then extracted two times with ethyl acetate and composite organic layer was collected and dried with NaCl and distilled to get bio-crude oil 2.58 gm (Yield>80%). The residue obtained after filtration was dried and the weight was noted to calculate percentage conversion (>85%). The calorific value of bio-crude oil from various MSW was 26 MJ/kg.

### (3) Production of Bio-Crude Oil Using Ionic Liquid Catalyst Synthesized in Example (1) (B) and Market Waste

The liquefaction reaction was conducted in batch mode operation in a 100 ml Amar reactor autoclave assembly having four peach bleded ampler and PID temperature controller with accuracy±1° C. The autoclave was loaded with 10 gm of crushed market MSW (H₂O content 70%) and [PEI⁺] [HSO₄⁻] ionic liquid catalyst (1.13 times w/w of dry MSW) and then pressurized at 20 bar N₂ pressure and then heated at 120° C. for 120 minutes at 400 rpm. After the completion of reaction, the reactor vessel was allowed to cool to room temperature before opening reactor vessel. Sufficient amount of water was added to the reaction mixture followed by vacuum filtration to remove unreacted MSW. The resulting filtrate was then extracted two times with ethyl acetate and composite organic layer was collected and dried with NaCl and distilled to get bio-crude oil 2.57 gm (Yield>80%). The residue obtained after filtration was dried and the weight was noted to calculate percentage conversion (>85%). The calorific value of bio-crude oil from various MSW was 25.8 MJ/kg.

### (4) Production of Bio-Crude Oil Using Ionic Liquid Catalyst Synthesized in Example (1) (B) and Cook Food Waste

The liquefaction reaction was conducted in batch mode operation in a 100 ml Amar reactor autoclave assembly having four peach bleded ampler and PID temperature controller with accuracy±1° C. The autoclave was loaded with 10 gm of cook food MSW (H₂O content 70%) and [PEI⁺] [HSO₄⁺] ionic liquid catalyst (1.13 times w/w of dry MSW) and then pressurized at 20 bar N₂ pressure and then heated at 120° C. for 120 minutes at 400 rpm. After the completion of reaction, the reactor vessel was allowed to cool to room temperature before opening reactor vessel. Sufficient amount of water was added to the reaction mixture followed by vacuum filtration to remove unreacted MSW. The resulting filtrate was then extracted two times with ethyl acetate and composite organic layer was collected and dried with NaCl and distilled to get bio-crude oil 2.58 gm (Yield>80%). The residue obtained after filtration was dried and the weight was noted to calculate percentage conversion (>85%). The calorific value of bio-crude oil from various MSW was 26.7 MJ/kg.

As can be observed from Example 2, 10 g of cook food waste and market waste was found to provide a minimum 25 MJ/kg. Usage of [PEI⁺] [HSO₄⁻] ionic liquid provided marginally higher calorific value than [BenzIMSO3H⁺] [HSO4⁻]. [PEI⁺] [HSO4⁻] ionic liquid catalyst was found to yield the highest calorific value bio-crude fuel with cook food waste. The corresponding calorific value was found to be 26.7 MJ/kg. In conclusion, the Example 1 provides an easy route that provides sulfonate-based ionic liquid in high yields (Yield>95%). Ionic liquids are dynamic polar molecules that are useful as catalyst and solvents. Additionally, as per the present invention, the sulfonate-based ionic liquids were found to be highly effective in recovering the useful carbon content of municipal solid waste by converting it into useful bio-crude fuel oil. Table 1 provides a summary of the general characteristics of the bio-crude fuel obtained by the process.

TABLE 1

| Sr. No | Entry | Results |
|---|---|---|
| 1 | Color | Dark brown |
| 2 | Density | 0.95-1.04 gm/mL |
| 3 | Calorific Value | 10-35 MJ/Kg |
| 4 | pH | 5-6 |
| 5 | Elemental composition | Carbon: 50%, |
| | (C, H, N, S and O) | Hydrogen: 6%, |
| | | Nitrogen: 0.8% |
| | | Sulfur: 0.22% |
| | | Oxygen: 43% |
| 6 | Ash content | 0.5-4% |
| | (by gravimetric analysis) | |
| 7 | Stability | Stable at normal storage |
| | | conditions |
| 8 | Chemical composition | Aromatics, hydrocarbons |
| | (by GC-MS) | (C₅-C₂₂), esters, alcohols, |
| | | phenols, aldehydes, ketones, |
| | | carboxylic acids (C₅-C₁₈) etc. |
| 9 | Flash Point | 66-70° C. |
| 10 | Pour point | 10-15° C. |
| 11 | Viscosity | 165-250 cpi |

As is clear from the values summarized in Table 1, the said catalytic liquefaction process could be highly useful in not only providing an alternate energy source, but also in reducing the toxic load of waste accumulation on the environment. The high flash point (66-70° C.), low ash content and high stability highlight the importance of the present invention. The most important point of the disclosed catalytic liquefaction process is the high calorific the remarkably high calorific value achieved (10-35 MJ/Kg).

Although the subject matter has been described in considerable details with reference to certain examples and embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the present subject matter as defined.

I/We claim:

1. An ionic liquid
selected from the group consisting of a compound of Formula I having viscosity in the range of 100-2000 cpi (centipoise) at 30° C. 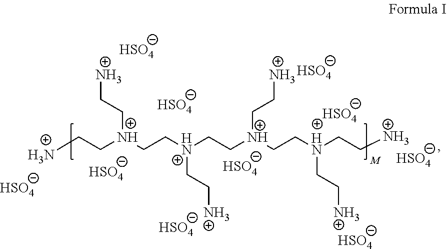
wherein M is number average molecular weight in the range of 0.2-150 KD; and
a compound of Formula II having viscosity in the range of 100-2000 cpi at 30° C. 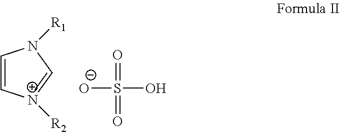
wherein R1 is selected from the group consisting of C₁ to C₁₀ alkyl and C₁ to C₂₀ aryl,
wherein the alkyl and aryl are optionally substituted with halo, —OH, aryl and alkyl;
R2 is selected from the group consisting of hydrogen, HSO₃⁻, C₁ to C₁₀ alkyl and C₁ to C₂₀ aryl, wherein the alkyl and aryl are optionally substituted with halo, —OH, aryl and alkyl.

2. An ionic liquid as claimed in claim 1, wherein the ionic liquid is represented by Formula I having viscosity in the range of 100-2000 cpi (centipoise) at 30° C. 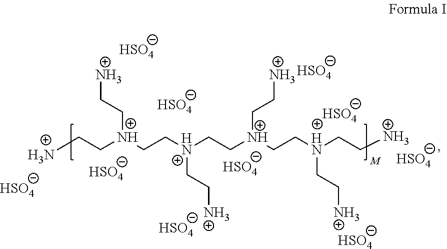
wherein M is number average molecular weight in the range of 0.2-150 KD.

3. An ionic liquid as claimed in claim 1, wherein the ionic liquid is represented by formula II having viscosity in the range of 100-2000 cpi at 30° C. 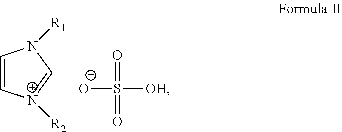
wherein R1 is selected from the group consisting of C₁ to C₁₀ alkyl and C₁ to C₂₀ aryl,
wherein the alkyl and aryl are optionally substituted with halo, —OH, aryl and alkyl;
R2 is selected from the group consisting of hydrogen, HSO₃⁻, C₁ to C₁₀ alkyl and C₁ to C₂₀ aryl, wherein the alkyl and aryl are optionally substituted with halo, —OH, aryl and alkyl.

4. An ionic liquid as claimed in claim 1, wherein the ionic liquid is represented by formula II having viscosity in the range of 100-2000 cpi at 30° C. 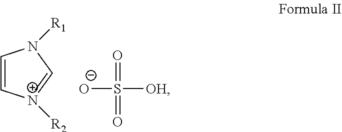
wherein R1 is selected from the group consisting of C₁ to C₃ alkyl, wherein the alkyl is optionally substituted with C₅ to C₁₀ aryl;
R2 is HSO₃⁻.

5. A process for obtaining the ionic liquid as claimed in any of the claims 1-4, comprising the steps of:
a) contacting at least one nitrogen substrate with at least one sulfonating agent and optionally at least one solvent to obtain a first mixture;
b) stiffing the first mixture to obtain the ionic liquid; and
c) isolating the ionic liquid

6. The process as claimed in claim 5, wherein the at least one nitrogen substrate is a compound of Formula III 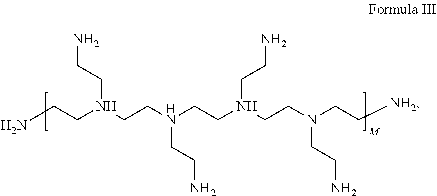
wherein M is number average molecular weight in the range of 0.2-150 KD.

7. The process as claimed in claim 5, wherein the at least one nitrogen substrate is a compound of Formula IV 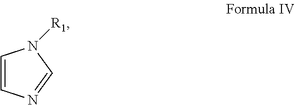
wherein R1 is selected from the group consisting of C₁ to C₁₀ alkyl and C₁ to C₂₀ aryl,
wherein the alkyl and aryl are optionally substituted with halo, —OH, aryl and alkyl.

8. The process as claimed in claim 5, wherein the at least one nitrogen substrate is a compound of Formula IV 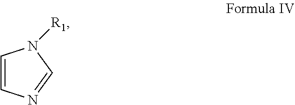
wherein R1 is selected from the group consisting of C₁ to C₃ alkyl, wherein the alkyl is optionally substituted with C₅ to C₁₀ aryl.

9. The process as claimed in claim 5, wherein at least one sulfonating agent is selected from a group consisting of chlorosulfonic acid, sulphuric acid, sulfur trioxide, and combinations thereof.

10. The process as claimed in claim 9, wherein at least one sulfonating agent is chlorosulfonic acid.

11. The process as claimed in claim 9, wherein at least one sulfonating agent is sulfuric acid.

12. The process as claimed in claim 5, wherein at least one solvent is selected from a group consisting of dichloromethane, ethylene dichloride, chloroform, carbon tetrachloride and combinations thereof.

13. The process as claimed in claim 5, wherein stiffing the first mixture is carried out at a temperature in the range of 30-150° C. at a stirring speed in the range of 400 rpm for a period in the range of 10-150 min.

14. The process as claimed in claim 6, wherein stiffing the first mixture is carried out at a temperature in the range of 30-80° C. at a stiffing speed in the range of 400 rpm for a period in the range of 10-150 min.

15. The process as claimed in claims 7 and 8, wherein stiffing the first mixture is carried out at a temperature in the range of 30-150° C. at a stiffing speed in the range of 400 rpm for a period in the range of 15-120 min.

16. The process as claimed in claim 5, wherein isolating the ionic liquid is carried out by a process selected from the group consisting of filtration, evaporation, distillation, solvent extraction and combinations thereof.

17. A process for obtaining a bio-crude fuel comprising the steps of:
a) contacting a waste with water to form a first mixture;
b) contacting the first mixture with at least one ionic liquid as claimed in claim 1-4;
c) heating the first mixture to obtain a second mixture;
d) processing the second mixture to obtain the bio-crude fuel; and
e) recovering the at least one ionic liquid from the second mixture to obtain recovered ionic liquid.

18. The process as claimed in claim 17 comprising the steps of:
a) contacting a waste with water to form a first mixture;
b) contacting the first mixture with at least one ionic liquid as claimed in claim 2;
c) heating the first mixture to obtain a second mixture;
d) processing the second mixture to obtain the bio-crude fuel; and
a) recovering the at least one ionic liquid from the second mixture to obtain recovered ionic liquid.

19. The process as claimed in claim 17 comprising the steps of:
b) contacting a waste with water to form a first mixture;
c) contacting the first mixture with at least one ionic liquid as claimed in claims 3 and 4;
d) heating the first mixture to obtain a second mixture;
e) processing the second mixture to obtain the bio-crude fuel; and
f) recovering the at least one ionic liquid from the second mixture to obtain recovered ionic liquid.

20. The process as claimed in any of the claims 17-19, wherein the said ionic liquid is a macromolecule having molecular weight in the range of 0.2-150 KD and the said process is effective in recovering the ionic liquid from second reaction mixture by a process selected from the group consisting of membrane separation, selective precipitation, adsorptive separation, distillation and combinations thereof.

21. The process as claimed in any of the claims 17-19, wherein the waste is a solid waste selected from the group consisting of municipal solid waste garbage, lignocellulosic biomass, algae, road swipe, kitchen waste, vegetable waste, cook food waste, paper waste, garden waste.

22. The process as claimed in any of the claims 17-19, wherein heating the first mixture is carried out at a temperature in the range of 30-200° C. at a stirring speed in the range of 300-450 rpm for a period in the range of 30-200 min.

23. The process as claimed in any of the claims 17-19, wherein the waste is optionally pre-processed by maceration, crushing, grinding, and combinations thereof.

24. The process as claimed in any of the claims 17-19, wherein the heating the first mixture is optionally carried out under pressure in the range of 0-50 bars.

25. The process as claimed in any of the claims 17-19, wherein the bio-crude fuel has a calorific value in the range of 10 MJ/Kg to 35 MJ/Kg.

26. The process as claimed in any of the claims 17-19, wherein the bio-crude fuel is obtained in a yield greater than 80%.

27. The process as claimed in any of the claims 17-19, wherein conversion of at least 85% is achieved.

28. The process as claimed in any of the claims 17-19, wherein the bio-crude fuel is used directly as a fuel or as a fuel-additive in automobile, industrial and agricultural applications.
